(19) United States
(12) Patent Application Publication
(10) Pub. No.: US 2003/0069634 A1
(43) Pub. Date: Apr. 10, 2003

(54) Two-step method to deposit and secure a chemical species coating onto a medical device

(76) Inventors: Susan Bialecke, San Francisco, CA (US); Charlene Chen, Redwood City, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 09/859,042

(22) Filed: May 16, 2001

(51) Int. Cl. A61F 2/06; A61L 2/00

(52) U.S. Cl. 623/1.42; 427/2.1; 427/5; 427/458; 427/569

(57) ABSTRACT

A two-step process for depositing and securing a chemical species onto a medical device. In the first step a chemical species is deposited onto a medical device. Electrodeposition is used in one technique. In the second steps, the deposited coating is secured to the medical device. In one technique, plasma recoil implantation is used. The two-step process is well-suited for non-metallic radioactive materials.

### FIELD OF THE INVENTION

The present invention relates to intravascular radiotherapy, and in particular, to a coating applied onto a medical device, such as a stent.

### BACKGROUND OF THE RELATED ART

A region of stenosis or atherosclerotic plaque build-up in an arterial lumen may reduce blood flow through the arterial lumen. To improve blood flow through a region of stenosis, the arterial lumen is typically treated in the region of the stenosis. Percutaneous transluminal coronary angioplasty (PTCA) is increasingly being used to improve blood flow by opening the occlusion of the arterial lumen at the region of stenosis.

PTCA generally involves delivering a distal end of an angioplasty catheter to the region of the stenosis inside the arterial lumen. A balloon at the distal end of the angioplasty catheter is positioned and then inflated to press against the region of the arterial lumen associated with the stenosis to reduce the restriction. Next, the balloon is deflated so the balloon and the catheter can be removed. Increased blood flow through the lumen results from this treatment.

However, after a PTCA procedure there is a chance that atherosclerotic plaque may return in a process known as restenosis. Restenosis can even result in a more severe build-up of plaque than in the original stenosis. To prevent or retard the restenosis, one technique implants a stent at the location of the treatment. The stent is typically a tubular device designed to expand and press against the arterial wall. To implant the stent, the stent is first secured over a balloon at the distal end of a balloon catheter. The balloon and stent are then advanced at the end of a catheter through the arterial lumen. At the region of stenosis, the balloon is inflated, expanding the stent against the arterial wall. The stent holds its expanded shape as the balloon is deflated and withdrawn. The expanded stent structure reduces the probability of an occurrence of the restenosis or reduces the severity of the restenosis.

Even with the stent implant, restenosis may still occur at the location of the stent. To prevent restenosis at the stent location, radiation is sometimes used to stunt cell growth in the arterial tissue near the region of the stent. In one form of radiation treatment, radioactive stents are used to both hold open the arterial lumen and deliver the radiation.

When placing the radioactive material onto a stent, care is taken to deposit a controlled coating onto the sent. Typically, a uniform coating is desired for a uniform dosage profile. However, in some instances, a controlled coating (other than a uniform coating) may be desired. Since consistent coating repeatability is difficult to achieve with some processes, it would be advantageous to have a more predictable process with consistent repeatability. Additionally, the process should ensure a secure coating so that the radioactive material does not separate from the stent and enter the blood stream.

Accordingly, the present invention provides for a two-step process to coat a chemical species on a medical device, such as a radioactive material onto a stent.

### SUMMARY OF THE INVENTION

This invention discloses a method of depositing a chemical species onto a surface of a medical device and securing the deposited chemical species on the medical device.

### BRIEF DESCRIPTION OF THE DRAWINGS

The present invention is illustrated by way of example and not limitation in the accompanying figures:

FIG. 1 shows one embodiment of a stent having a coating embodying a practice of the present invention.

FIG. 2 shows one embodiment of an apparatus that can be used to react a chemical species with material on a surface of a medical device to practice the invention.

FIG. 3 shows one embodiment of a holder to hold a stent in order to subject the stent to implantation to secure a deposited chemical species.

FIG. 4 shows one embodiment of an apparatus that can be used to electrodeposit a chemical species onto a surface of a medical device to practice the invention.

FIG. 5 shows another embodiment of an apparatus that can be used to electrodeposit a chemical species onto a surface of a medical device to practice the invention.

### DETAILED DESCRIPTION OF THE INVENTION

The following description makes reference to numerous specific details in order to provide a thorough understanding of the present invention. However, it is to be noted that not every specific detail need be employed to practice the present invention. Additionally, well-known details, such as particular materials or methods, have not been described in order to avoid obscuring the present invention.

An embodiment of the present invention is a two-stage process to deposit a non-metal chemical species onto a medical device and then to secure the deposited coating to the medical device.

In step one, a chemical species, such as radioactive phosphorus, is deposited onto a medical device, such as a stent. In the described embodiment, a non-metal chemical species is deposited. In one embodiment of the invention, a coating of the non-metal chemical species may be deposited on the medical device by immersing the medical device in a solution containing the non-metal chemical species. The non-metal chemical species may then react with and coat the medical device. In another embodiment of the invention, a current may be applied through the medical device in the solution containing the non-metal chemical species to electrodeposit a coating of the non-metal chemical species on the medical device. Other methods of coating the medical device with a non-metal chemical species not described herein are also within the scope of the invention.

In step two, the deposited chemical species is secured to the medical device. Because the deposited coating may separate from the medical device prior to, during, or after a medical procedure, the deposited chemical species is securely affixed onto the medical device. When the deposited chemical species is non-metal, there is more likelihood that the material will flake off unless the second step is performed. Various methods may be used to secure the deposited coating to the medical device, including, but not limited to, plasma recoil implantation, plasma implantation, plasma coating, or applying a second material coating over the deposited chemical species. For example, a second material coating can be applied over the deposited chemical species by electrodepositing a metal or applying a polymer coating. Other methods of securing the chemical species to the medical device not described herein are also within the scope of the invention.

It is appreciated that a wide variety of medical devices, including intravascular or intraductal stents and radiotherapy sourcewires, can be treated by the practice of the invention. That is, the invention is not limited to stents alone. In one embodiment, the stent, or any other medical device for that matter, to be coated is metallic or at least partially metallic. The medical device may be made of titanium, stainless steel, nitinol, gold, chromium, iron, nickel, or other biocompatible metals or metal alloys.

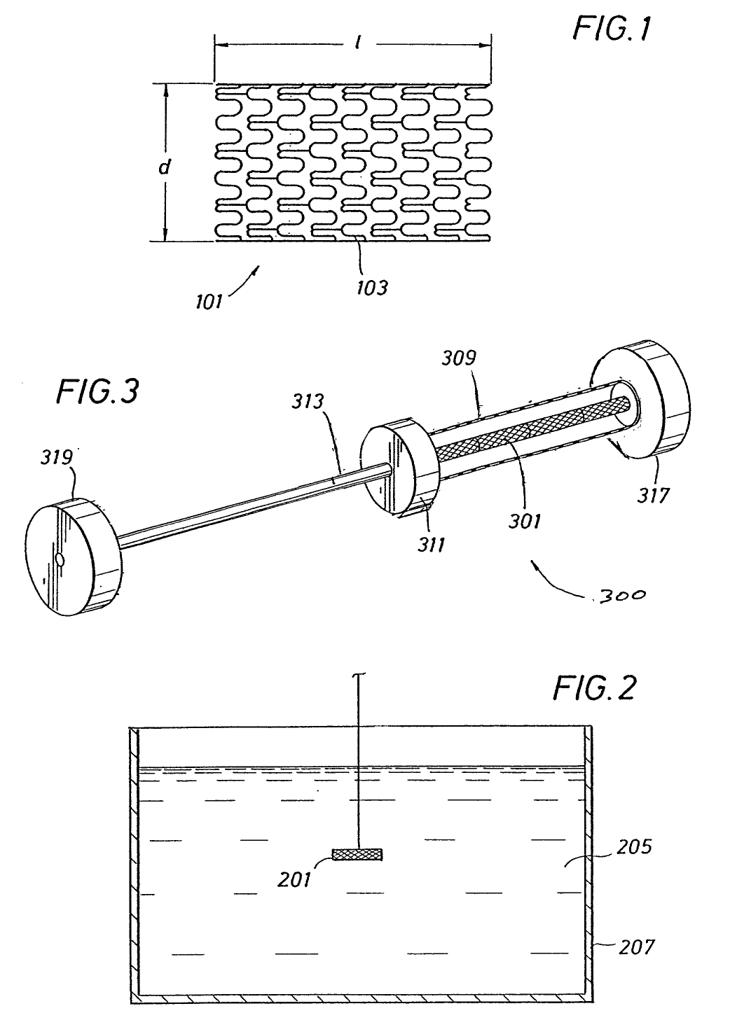

As shown in FIG. 1, the particular medical device to be processed with the two-step method is a stent 101. The particular stent embodied is constructed of thin-walled cylindrical shaped structures formed of interconnecting cylindrical elements 103. One of a variety of prior art stents can be adapted for the stent 101, if desired. Although dimensions are not critical, one embodiment of the stent 101 has a length in the range of approximately 5 mm to 100 mm. Generally, the stent sizing and length will be dependent on the vasculature and the length of the stenosis. The interconnecting cylindrical elements 103 can be designed to permanently deform when expanded inside a body lumen. Expanded stent diameter d may be in the range of approximately 2 mm to 12 mm for one embodiment, although other diameters can be used depending on the diameter of the vasculature, etc. Once permanently deformed, the stent 101 generally holds its shape, thereby maintaining an open passage through the body lumen.

In one embodiment of the invention, the medical device to be coated is a partially metallic stent, and the non-metal chemical species to be deposited is radioactive phosphorus. As shown in FIG. 2, to deposit the radioactive phosphorus onto the stent 201, the stent 201 is lowered into a solution 205 of radioactive phosphoric acid disposed in a container 207. A natural reaction known as phosphating may take place between the metal on the stent 201 and the radioactive phosphorus, ³²P in the solution 205. For example, if the stent 201 has iron present, either of the following phosphating reactions may take place to produce a radioactive phosphorous in the form PO₄⁻³.

2 Fe+2 H₃PO₄→2 FePO₄+3 H₂↑

Fe₂O₃+2 H₃PO₄→2 FePO₄+3 H₂O

Under the chemical reaction the FePO₄ adheres to the surface of the stent 201. Generally, the stent 201 may be left in the solution 205 for anytime within the range of approximately 2 minutes to 2 hours. In one embodiment, the stent 201 remains in the solution 205 for approximately one hour. Typically, longer duration results in a thicker deposited coating of radioactive phosphorus on the stent 201. Because the deposited coating of radioactive phosphorus may only be holding onto the stent 201 through adhesion, the deposited coating of radioactive phosphorus is secured to the stent 201 by the second phase of the process to prevent the coating from being removed from the surface of the stent 201 in the securing step described later.

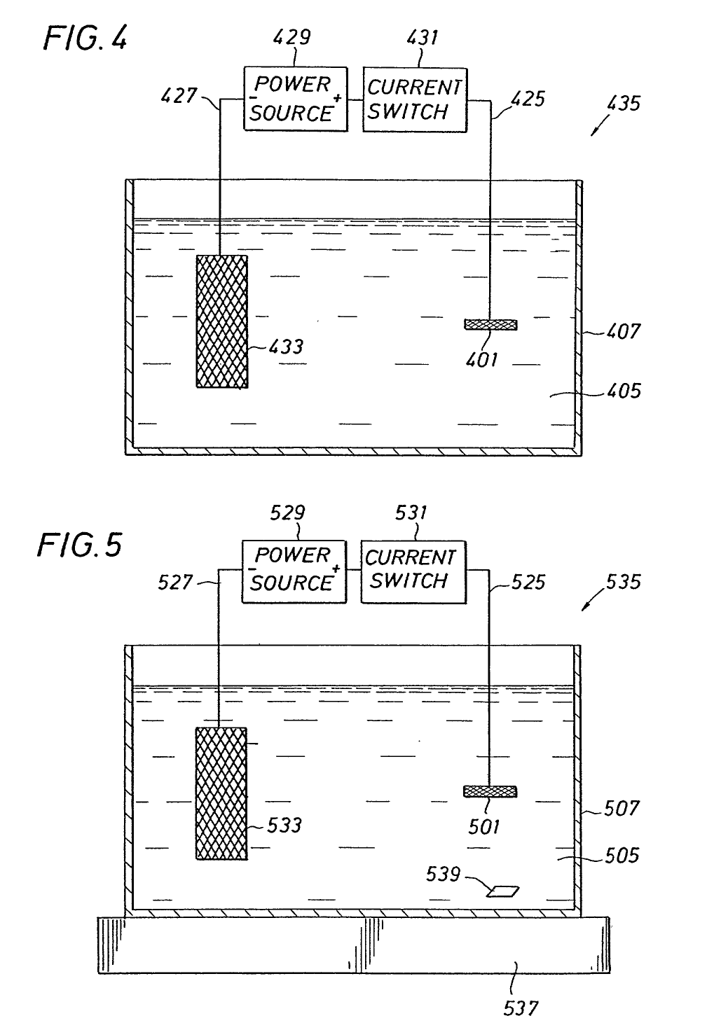

In another embodiment of the invention, the non-metal chemical species may be electrodeposited on the medical device during the first step. For example, as seen in FIG. 4, the example medical device to be coated is a stent 401, and the non-metal chemical species to be deposited is again radioactive phosphorus. In FIG. 4, an apparatus 435 for electrodepositing a chemical species on an implantable medical device, such as the stent 401 is shown.

In the embodiment shown in FIG. 4, a power source 429 is used to apply a current through an immersed stent 401. The stent 401 is coupled to, or in close proximity to, an anode electrode 425 that is coupled to the power source 429. An electrode of the power source 429 is attached to a conductive mesh screen 433 In this example, the electrode is a cathode electrode 427. The conductive mesh screen is utilized to provide a more even current flow through the stent 401. Alternately, depending on the chemical species to be electrodeposited, the electrode polarities on the conductive mesh screen 433 and the stent 401 may be reversed. The actual polarities used will depend on the chemistries of the material to be deposited.

The stent 401 and the conductive mesh screen 433 may be coupled to the corresponding electrodes 425 and 427 of the power source 429 by various methods. The stent 401 and the conductive mesh screen 433 may be attached directly, such as by wires or clips. The stent 401 may also be mounted onto a fastening mechanism such as a mandrel that is then attached to the anode electrode 425. The mandrel may then be lowered into the liquid solution 405 with the conductive mesh screen 433. Care may be needed to minimize the surface area of the stent 401 covered by any part of the fastening mechanism. Covered parts of the stent 401 may not be coated during electrodeposition.

The conductive mesh screen 433 and the stent 401 may be immersed in a liquid solution 405 typically comprised of water, such as distilled water, and the chemical species to be electrodeposited, such as radioactive phosphoric acid. The concentration of the liquid solution 405 is in the range of approximately 5 to 15 mCi/mL, for one embodiment. In one embodiment, the solution has a concentration of 10 mCi/mL. Other concentrations may be utilized. The liquid solution 405 is generally contained in an insulative container 407. However, other containers may be used. If the liquid solution 405 is radioactive, necessary precautions may be needed in handling the apparatus 435 and the liquid solution 405.

A current switch 431 connected on either side of the power source 429 can be used to switch on the current. In one embodiment, a current in the range of approximately 1.0 to 500 mA flows through the stent 401 and the liquid solution 405. In another embodiment, a current of 5 mA is used. The concentration level and the current affect the deposition cycle in controlling the amount or the time required to controllably deposit a coating of the radioactive material onto the stent 401. Applying the current can speed up the reaction by removing electrons from the stent 401 to make the metal on the stent 401 more attractive to PO₄³⁻ ions in the radioactive phosphoric acid solution.

If radioactive phosphorus is used as the non-metal chemical species in the liquid solution 405, a voltage in the range of approximately 20 to 500 volts may be applied between the anode electrode 425 and the cathode electrode 427. In one embodiment, a voltage of approximately 100 volts is used. Other currents and voltages may be used for different deposited coating depths or for different chemical species. The power source 429 may have a fixed voltage, or optionally, have a user modifiable voltage. With one embodiment the current and the voltage are set at predetermined levels noted above for a time in the range of approximately 2 minutes to 1 hour. In one particular embodiment, an approximate time of 30 minutes is used. Depending on the voltage and the current selected, other times may be used.

During electrodeposition, the current may start to drop as the coating process progresses. Therefore, it may be necessary to increase the voltage during the electrodeposition process to maintain a set current. Furthermore, instead of increasing the voltage, the stent 401 may be electrodeposited for a longer time at the lower current.

Referring to FIG. 5, another embodiment that is equivalent to the embodiment of FIG. 4 is shown. In this embodiment, while the voltage is applied, a liquid solution 505 is agitated to displace hydrogen gas bubbles forming on a stent 501. Alternatively, the stent 501 can be agitated. For example, a stir bar 539 may be rotated in a container 507 by a magnetic stirring plate 537. The rotating magnet will move the stir bar 539 around in the container to agitate the liquid solution. By agitating the solution to displace the hydrogen bubbles that build up on the stent 501 during electrodeposition, the electrodeposition process may be sped up to allow for a more even coating on the stent 501 surface. Also, a current switch 531 may be attached to either side of a power source 529 to moderate current through the stent 501 and conductive mesh screen 533.

It is to be noted that other agitating devices can be employed. Furthermore, to speed up the electrodeposition process, oxidizing agents may be added to the solution. The oxidizing agents may absorb hydrogen that otherwise would have formed hydrogen bubbles on the stent surface. Oxidizing agents may be added in addition to agitating the solution or may be used alone. Absorbing the hydrogen may result in a more controlled deposition.

It is appreciated that any one of the techniques described above in reference to FIGS. 2, 4 and 5 can be used to deposit a radioactive species onto the medical device. Stents 201, 401 and 501 are used as examples of a medical device. The deposition techniques allow for a controlled deposition of the radioactive species.

In many instances, a uniform coating is desired on the medical device. However, in some instances, a radiation profile other than uniform is desired. For example, a hot or cold-end stent may be desired where the radiation dose profile at the ends may be more (hot-end) or less (cold-end) than the center area of the stent. In such cases, various masking techniques can be employed for more/less coating at selected locations. The step one techniques described above allow for a more reliable and repeatable controlled deposition of a radioactive species on a medical device, such as a stent. It also allows for a more concentrated deposit, as well.

After depositing a non-metal chemical species onto the stent 201, 401, or 501, in step one, the second step of securing the electrodeposited coating to the stent is performed. Several processes may be used in step two to secure the electrodeposited coating to the stent including but not limited to plasma recoil implantation, plasma implantation, plasma coating, electrodepositing a second coating over the electrodeposited non-metal chemical species, and applying a polymer coating over the electrodeposited non-metal chemical species. Other methods of securing the non-metal chemical species to the medical device not described herein are also within the scope of the invention.

After the completion of the controlled deposition process of the first step, the second step is used to secure the chemical species onto the medical device. As noted, one of the above described securing techniques, as well as others, insures that the deposited chemical species is affixed securely on the medical device. Without this second securing step, there is an increased likelihood that the chemical species will separate from the medical device, especially when the chemical species is non-metal. In the instance where radioactive material is deposited onto a stent, securing the radioactive material is imperative for patient welfare. One particular process for securing deposited chemical species is the utilization of plasma recoil implantation. Plasma recoil implantation uses high-energy plasma to embed the deposited coating into the surface of the medical device. For example radioactive phosphorous can be plasma recoil implanted into a stent by striking molecules of the deposited coating of radioactive phosphorous.

Generally, once the chemical species is deposited onto the medical device, the medical device is placed in a plasma chamber and subjected to plasma recoil implantation. Although a variety of plasma implantation techniques could be utilized, plasma recoil implantation has specific advantages in that the higher energy levels imbeds the molecules of the chemical species into the surface of the medical device to ensure that the chemical species will not readily separate from the medical device.

FIG. 3 shows one example apparatus for holding a stent, or a plurality of such stents 301, for placement in a plasma recoil implantation chamber. FIG. 3 shows one embodiment of a fixture 300 for holding a plurality of stents 301. The stents 301 are loaded onto a support rod 313, in which the stents 301 are loaded by placing them end to end on the support rod 313. After loading the stents 301 onto the support rod 313, a cylindrical mesh 309 may be secured over the stents 301 using two endcaps 311 and 317. The cylindrical mesh 309 is used to prevent thermal damage from plasma that strikes the molecules of the deposited coating on the stent 301 surface. It is to be noted that the plasma recoil implantation technique can be used without the presence of the cylindrical mesh 309. When used, the cylindrical mesh 309 is typically made of titanium or tantalum although other metals can be used. A separate endcap 319 is disposed at the other end of the support rod 313 so that caps 317 and 319 are used as encaps at both ends of the support rod 313. Again the inner cap 311 and the exterior cap 317 are utilize to hold the stents 301 in place, as well as the cylindrical mesh 309, when used.

Once the stents 301 are loaded in position within the fixture 300, the fixture 300 is placed in a plasma chamber and subjected to plasma recoil implantation. In one embodiment, ion voltage in the approximate range of 20 keV to 60 keV is applied between two electrodes in the plasma chamber. One of the electrodes is typically coupled to the support rod 313 so that the stents 301 effectively operates at one electrode end. In one particular embodiment an approximate ion voltage of 50 keV is applied between the two electrodes. The time required to implant the deposited chemical species into the medical device will be dependent on the various parameters of the reactor chamber as well as the chemical species being implanted. Generally an approximate range of 1 to 10 minutes is sufficient to perform the securing step. In one embodiment the high voltage is applied for approximately 5 minutes.

The high voltage of the chamber causes the plasma in the chamber to strike the stent surface at a sufficient velocity to secure the chemical species onto the surface of the medical device. Again, with the examples shown, radioactive material, such as radioactive phosphorous, is securely affixed to the stents 301. It is to be noted that a variety of plasmas can be utilized to perform the recoil implantation, provided the plasma material is compatible with the chemical species deposited on the medical device.

It is to be noted that the invention is not limited to depositing a non-metal chemical species. In some applications metallic chemical species may be deposited on the surface of the medical device. Examples of radioactive chemical species that may be used are radioactive phosphorus (such as ³²P), yttrium-90, and radioactive gold (Au-198), as well as others. Furthermore, the radioactive chemical species to be deposited in step one of the invention may have radioactive ions that are alpha emitting, beta emitting, or gamma emitting depending on the desired type of medical treatment.

The two-step process of depositing a coating on a medical device and then securing the deposited coating to the medical device insures more consistent controllable coating to be deposited and securely affixed. The two-step process is well suited for non-metallic chemical species.

Although an exemplary embodiment of the invention has been shown and described in the form of a coated medical device, many changes, modifications, and substitutions may be made without departing from the spirit and scope of this invention.

We claim:

1. A method comprising:
depositing a chemical species on a medical device; and
securing the deposited chemical species onto the medical device.

2. The method of claim 1 wherein the chemical species is a non-metal.

3. The method of claim 1 wherein the chemical species is radioactive.

4. The method of claim 3 wherein the chemical species is selected from a group consisting of radioactive phosphorus, radioactive yttrium, and radioactive gold.

5. The method of claim 1 wherein said securing the deposited chemical species is secured by plasma recoil implantation.

6. The method of claim 1 wherein the chemical species is deposited on a surface of the medical device by electrodeposition.

7. A method comprising:
depositing a non-metal chemical species onto a surface of a medical device; and
embedding the deposited non-metal chemical species into the surface of the medical device by implantation.

8. The method of claim 7 wherein the chemical species is radioactive.

9. The method of claim 7 wherein the non-metal chemical species is deposited by electrodeposition.

10. The method of claim 8 wherein the nonmetal chemical species is radioactive phosphorus.

11. The method of claim 8 further comprising immersing the medical device in a solution containing of the non-metal chemical species to deposit the chemical species.

12. The method of claim 11 wherein the non-metal chemical species is deposited by electrodeposition.

13. The method of claim 11 wherein the solution concentration is in the approximately range of 5 to 15 mCi/mL.

14. The method of claim 8 wherein the embedding is achieved by plasma implantation.

15. The method of claim 5 wherein the embedding is achieved by plasma recoil implantation.

16. The method of claim 15 wherein an ion voltage in the approximately range of 20 to 60 keV is applied to a plasma used for the plasma recoil implantation.

17. An apparatus comprising:
a medical device; and
a chemical species secured on said medical device by implantation after having said chemical species deposited thereon.

18. The apparatus of claim 17 wherein said chemical species is radioactive.

19. The apparatus of claim 17 wherein said medical device is a stent.

20. The apparatus of claim 17 wherein said chemical species is a non-metal.

21. The apparatus of claim 17 wherein said chemical species is implanted by plasma implantation.

22. The apparatus of claim 17 wherein said chemical species is implanted by plasma recoil implantation.

23. The apparatus of claim 17 wherein said chemical species is a non-metal radioactive species and secured by plasma recoil implantation.

24. The apparatus of claims 23 wherein said medical device is a stent.
